(19) United States
(12) Patent Application Publication
(10) Pub. No.: US 2020/0046993 A1
(43) Pub. Date: Feb. 13, 2020

(54) MAGNETIC HEALING SYSTEM AND METHOD OF USE

(71) Applicant: Hirofusa Otsuba, Middle Village, NY (US)

(72) Inventor: Hirofusa Otsuba, Middle Village, NY (US)

(21) Appl. No.: 16/100,444

(22) Filed: Aug. 10, 2018

(51) Int. Cl. A61N 2/06; A61N 2/00

(52) U.S. Cl. CPC A61N 2/06; A61N 2/004

(57) ABSTRACT

A magnetic healing system uses two like magnets arranged so that a similar pole from each is aligned and thus produces free electrons. The free electrons are able to enter a person's body and neutralize free radicals found there. The aligned magnets are part of the free electron generator and are held in place by a body.

### BACKGROUND

### 1. Field of the Invention

The present invention relates generally to healing systems, and more specifically, to a magnetic healing system for improving the rate at which a body can recover or mend from a wound or illness.

### 2. Description of Related Art

Healing systems are well known in the art and are effective means to promote or improve the wellbeing of people or other living creatures through treatments, medications, training or the like. For example, FIG. 1 depicts a conventional bandage system 101 having gauze pad 105 held against a wound 107 by protective tape 103 having an adhesive side 109. The pad 105 prevents contaminates from entering the wound 107 and fluids from exiting the wound 107. It is also common to place ointments or medications between the pad 105 and wound 107.

One of the problems commonly associated with system 101 is its limited efficiency. For example, the pad 105 does keep the wound clean to promote healing there are no active effects on the healing from the pad 105. The healing time is completely dependent on person being treated.

Accordingly, although great strides have been made in the area of bandage systems, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a side view of a common bandage system;

FIG. 2 is a cross-sectional side view of a magnetic healing system in accordance with a preferred embodiment of the present application;

FIG. 3 is a perspective view of the free electron generator of FIG. 2;

FIG. 4 is a perspective view of the free electron generator of FIG. 2;

FIG. 5 is a top view of an alternative embodiment of the system of FIG. 2;

FIG. 6 is a top view of an alternative embodiment of the system of FIG. 2;

FIG. 7 is a cross-sectional side view of an alternative embodiment of the system of FIG. 2;

FIG. 8 is a cross-sectional view of an alternative embodiment of the system of FIG. 2; and

FIG. 9 is a flow chart of the preferred method of use of the system of FIG. 2.

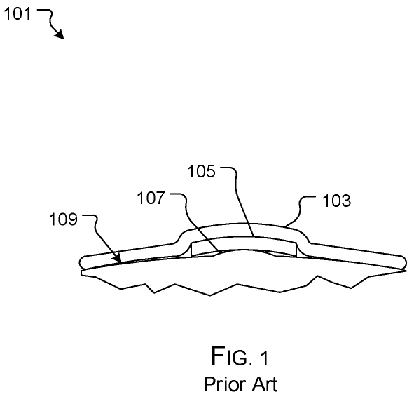

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional bandage systems. Specifically, the invention of the present application encourages healing by introducing free electrons to a treatment area. This and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

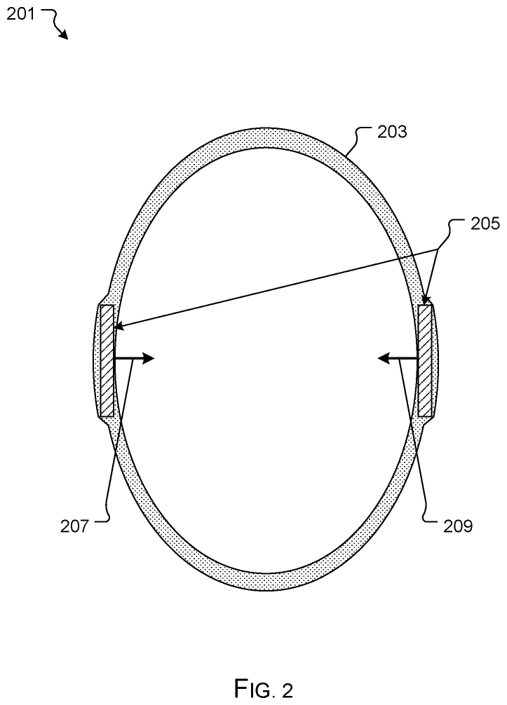

Referring now to the drawings wherein like reference characters identify corresponding or similar elements throughout the several views, FIG. 2 depicts a cross-sectional side view of a magnetic healing system in accordance with a preferred embodiment of the present application. It will be appreciated that system 201 overcomes one or more of the above-listed problems commonly associated with conventional bandage systems.

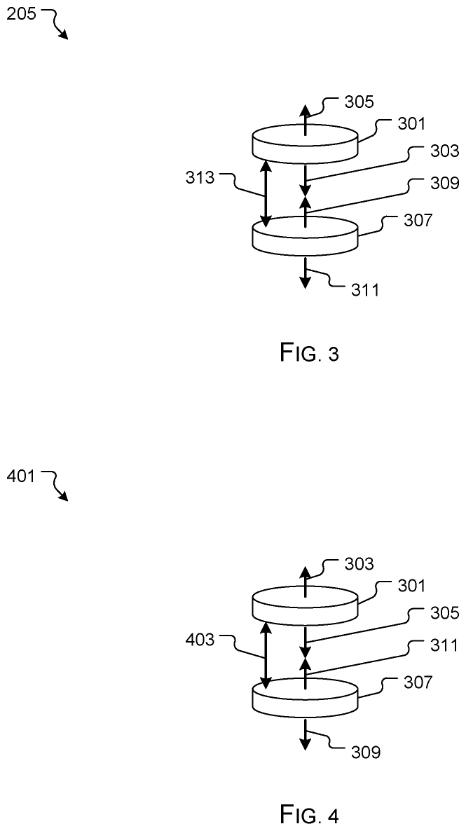

In the contemplated embodiment, system 201 includes a body 203 wherein a free electron generator 205 is housed. The body 203 configured to attach to a user's body. In the current embodiment the body 203 is configured as a band. The free electron generator 205 includes a first like pole 207 aligned with a second like pole 209 as will be further described in FIG. 3.

As depicted in FIG. 3 the free electron generator 205 includes a first magnet 301 with a first north pole 303 and a first south pole 305 and a second magnet 307 with a second north pole 309 and a second south pole 311. The first magnet 301 being held in proximity to the second magnet 307 so that the first north pole 303 is adjacent to the second north pole 309 thus inducing free electrons in an interstitial space 313.

It should be appreciated that one of the unique features believed characteristic of the present application is that the interaction between the first north pole 303 and the second north pole 309 create and release free electrons to assist with the healing of the body by neutralizing free radicals found therein.

Referring now to FIG. 4, an alternative configuration of the free electron generator 205. Embodiment 401 including similar features and components as those of the free electron generator 205 in FIG. 3 wherein the first south pole 305 and second south pole 311 are held in proximity to create a field of free electrons in interstitial space 403. It will be appreciated that either the north or south poles of the magnets are able to produce the field of free electrons so long as they are so oriented.

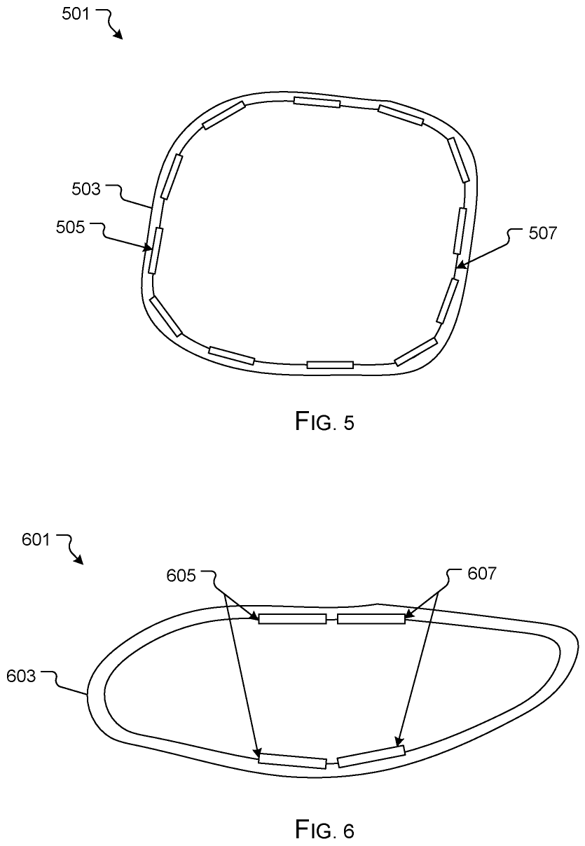

Alternative embodiments of the body 203 of system 201 are depicted by FIGS. 5 and 6. Embodiment 501 including a body 503 configured to cover a large area with free electron generators 505 arranged around the inner surface 507 thereof. Embodiment 601 including a body 603 configured to focus on a narrow treatment area with free electron generators 605 and 607.

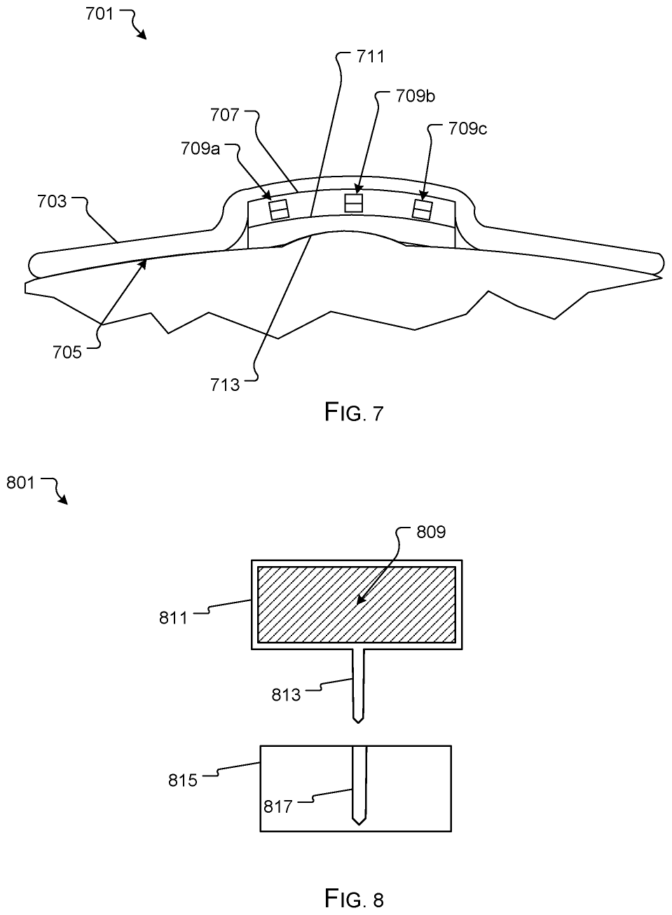

Referring to another alternative embodiment of system 201 is depicted by FIG. 7. Embodiment 701 including a body 703 having an adhesive side 705 with a protective pad 707 attached thereto. Pad 707 enclosing free electron generators 709 therein. The protective pad 707 having an absorbent pad 711 attached thereto. The absorbent pad 711 configured to be placed next to a wound 713.

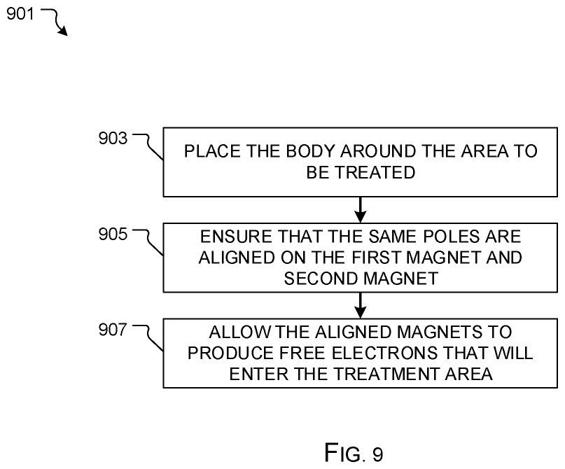

Referring to another alternative embodiment of system 201 is depicted in FIG. 8. Embodiment 801 including a body 811 enclosing free electron generator 809 having a pin 813 with a pin housing body 815 and pin hole 817 therein.

It will be appreciated that the embodiment 701 combines the benefits of a bandage system 101 with that of the magnetic healing system 201 to further improve the efficiency thereof. It will be noted that the free electron generators 709 have no interstitial space between the magnets thereof. The inclusion of the interstitial space it to better control the activity of the free electrons.

The preferred method of use of the system 201 is depicted in FIG. 9. Method 901 including placing the body around the area to be treated 903, ensuring that the same poles are aligned for the first magnet and second magnet 905 and allowing the aligned magnets to produce free electrons that will enter the treatment area 907.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed:

1. A magnetic healing system comprising:
a body configured to hold the system on a user; and
at least one free electron generator;
the free electron generator comprising a first magnet and a second magnet aligned so that the first north pole of the first magnet is opposite the second north pole of the second magnet;
wherein the free electrons resulting of the interaction of the first north pole and the second north pole are able to exit the first magnet and second magnet and enter the user.

2. The system of claim 1 wherein the first south pole of the first magnet and the second south pole of the second magnet are aligned to produce the free electrons.

3. The system of claim 1 wherein the body is configured to attached via fasteners or adhesive to the user.

4. The system of claim 1 wherein the body is configured to be placed around the neck, torso, head or appendages of the user.

5. The system of claim 1 wherein the first magnet and second magnet are joined together with the poles aligned.

6. The system of claim 1, wherein the body includes a pin with a pin housing configured to receive the pin therein.

7. The method of neutralizing free radicals given the system of claim 1, comprising:
placing the body around the area to be treated;
ensuring that the same poles are aligned for the first magnet and second magnet; and allowing the aligned magnets to produce free electrons that will enter the treatment area.
